(19) United States
(12) Patent Application Publication
(10) Pub. No.: US 2026/0354747 A1
(43) Pub. Date: Nov. 12, 2026

(54) A DEVICE AND SYSTEM FOR MONITORING CHEWING HABIT AND A METHOD THEREOF

(71) Applicants: Olive KHURANA, Greater Noida, Uttar Pradesh (IN); Olina KHURANA, Greater Noida, Uttar Pradesh (IN)

(72) Inventors: Olive KHURANA, Greater Noida, Uttar Pradesh (IN); Olina KHURANA, Greater Noida, Uttar Pradesh (IN)

(21) Appl. No.: 19/130,797

(22) PCT Filed: Nov. 17, 2023
(86) PCT No.: PCT/IB2023/061638

§ 371 Date: May 16, 2025

(30) Foreign Application Priority Data
Nov. 21, 2022 (IN) 202211066622

(51) Int. Cl. A61B 5/00; A61B 5/11

(52) U.S. Cl. CPC A61B 5/4542; A61B 5/0004; A61B 5/1123; A61B 5/4205; A61B 5/486; A61B 5/6814; A61B 5/7267; A61B 5/746; A61B 2560/0412; A61B 2562/0219

(57) ABSTRACT

System 100 and method for monitoring chewing habit includes a motion sensing device 102 affixed to an exterior part of face of a user 106, and a computing device 104 in communication with the motion sensing device and having a processor. The motion sensing device 102 is configured to detect jaw movement and transmit data corresponding to the jaw movement to the computing device 104. The computing device is configured to determine, from the received data, if the detected jaw movement pertains to a chewing action, determine a number of consecutive chewing actions by the user, and provide a feedback to the user 106 in respect of his chewing habit.

### TECHNICAL FIELD

The present disclosure relates to the field of devices and system for monitoring human behaviour. In particular, it pertains a device and system and method for monitoring chewing habits of a person.

### BACKGROUND

Background description includes information that may be useful in understanding the present invention. It is not an admission that any of the information provided herein is prior art or relevant to the presently claimed invention, or that any publication specifically or implicitly referenced is prior art.

Digestion of solid and semi-solid food begins by breaking down a morsel (also referred to as bite, hereinafter) into smaller pieces under repeated crushing action of teeth, a process known as chewing. Chewing is essential for improving efficacy of digestion process. It is recommended that food, before swallowing, should be turned into a semi-liquid form mixed properly with saliva to ensure proper digestion.

Another advantage of adequate chewing is that it prolongs the eating session, which gives sufficient time for brain to register signals from stomach and generate a signal corresponding to satiation for the person to stop eating. A faster food intake on account of in adequate chewing results in overeating and corresponding adverse effects, such as lifestyle diseases.

However, many a times, such as when eating in a hurry, or under stress, or by habit, food is not chewed sufficiently before swallowed. This leads to adverse effects on digestion, and leads to overeating, which in some cases, also leads to lifestyle diseases.

In spite of known advantages of proper chewing, it is difficult for a person to ensure that he chews each morsel sufficient number of times. This is essentially because chewing is by habit, and habits are difficult to change, unless constantly reminded. Therefore, in order to change the chewing habit, it is imperative to have a system that can monitor chewing of each morsel while eating so as to provide feedback on the quality of chewing activity to a user.

Patent documents WO2021131906A1 and US20220323189A1 disclose a jaw movement analysis system that includes circuitry that is configured to acquire chewing information including time-series information that represents a jaw movement of a user chewing a bite of food, and to determine an attribute of the food having been chewed by the user based on the chewing information acquired and based on an analysis model. The system includes a denture having a sensor to perform a function of acquiring measurement data (jaw movement information) relating to the jaw movement of the user during eating.

As can be seen the cited references disclose a system for monitoring the chewing habits of a person that involves wearing a denture, which can be inconvenient & cumbersome and may not be possible for everyone.

There is therefore a need in tea art of a simple system and method for monitoring chewing habit of a person that can be implemented easily and helps a user to improve his chewing habit.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### OBJECTS OF THE INVENTION

An object of the present invention is to provide a device, system and method for monitoring chewing habit of a person.

Another object of the present invention is to provide a device, system and method that helps a user to improve his chewing habit.

Another object of the present invention is to provide a device that helps in detecting chewing action of a user.

Another object of the present invention is to provide a system that helps in detecting number of times a morsel is chewed.

Another object of the present invention is to provide a system that provides an instant feedback to user on any deviation from a desired chewing habit.

Another object of the present invention is to provide a system that provides an feedback on change in his habit over a period of time.

Another object of the present invention is to provide a system that can be implemented with a portable device of the user, such as a smartphone.

### SUMMARY

Aspects of the present disclosure relate to a system and method for monitoring chewing habit of a person. Specifically, the present disclosure provides a system and method for monitoring chewing action of a user and providing him feedback, both instantly when a deviation from a desired chewing habit is detected, as well as on a long term bases on change in his habit over a period of time. Thus the disclosed system and method can help the user to change his chewing habit.

In an aspect, the disclosed device for monitoring chewing habit is configured to be removably affixed to an exterior part of face of a user, and is configured to, through motion sensing, detect jaw movement of the user while the user chews a food item, and transmit one or more data packets corresponding to the detected jaw movement to a remote device.

In an embodiment, the remote device may be a computing device configured to process the received one or more data packets and evaluate, for the user, any or a combination of chewing pattern, chewing frequency, number of chews, timespan of chewing associated with one or more bites of said food items.

In an embodiment, the remote device may implement a machine learning algorithm to evaluate chewing trends of said user and/or of a plurality of other users over a duration of time to indicate one or more chewing recommendations to the user.

Another aspect of the present disclosure relates to a system for monitoring chewing habit that includes a motion sensing device and a computing device in communication with the motion sensing device. The motion sensing device is affixed to an exterior part of face of a user, and is configured to detect jaw movement and transmit a data packet corresponding to the detected jaw movement. The computing device is in communication with the motion sensing device and includes a processor coupled with a memory, wherein the memory stores one or more instructions executable by the processor to: receive, from motion sensing device, the data packet corresponding to the detected jaw movement; determine, from the received data packet corresponding to the detected jaw movement, if the detected jaw movement pertains to a chewing action; determine, based on the determined chewing action, a number of consecutive chewing actions by the user; and provide a feedback to the user in respect of his chewing habit.

In an embodiment, the motion sensing device may be configured as a patch adapted to be affixed to a lower chin portion of the face of the user.

In an embodiment, the motion sensing device may be configured for being removably affixed to the face of the user.

In an embodiment, the motion sensing device may include any or a combination of an accelerometer and a gyro-sensor to detect the jaw movements, and a wireless transmitter to transmit the data packet corresponding to the detected jaw movement. In an embodiment, the processor may be configured to detect at least one of a swallowing action and a food intake action of the user.

In an embodiment, the processor may be configured to detect the chewing action, the swallowing action and the food intake action based on pattern of the jaw movement.

In an embodiment, the pattern of jaw movement may include one or more of a direction of movement, a duration of movement, a distance of movement, a duration between consecutive movements in the same direction, a time gap between movements in the opposite directions.

In an embodiment, the processor may be configured to provide a warning when it is detected that the number of chewing actions between consecutive food intake actions, or between a food intake action and a swallowing action is less than a predefined numbers.

In an embodiment, the processor may be configured to provide an output in respect of historical data including the number of chewing actions between consecutive food intake actions, or between a food intake action and a swallowing action.

In an embodiment, the computing device may be any of a mobile phone or a tablet of the user, and functionalities of the system may be implemented in an app.

Yet another aspect of the present disclosure relates to a method for monitoring chewing habit of a user. The method includes the steps of; (i) affixing a motion sensing device to an exterior part of face of a user, the motion sensing device being configured to detect jaw movement; (ii) transmitting, using a transmitter configured with the motion sensing device, data corresponding to the detected jaw movement to a computing device of the user; (iii) determining, at the computing device, from the received data corresponding to the detected jaw movement, if the detected jaw movement pertains to a chewing action; (iv) determining, based on the determined chewing action, a number of consecutive chewing actions by the user; and (v) providing a feedback to the user in respect of his chewing habit.

In an embodiment, the method may further include the step of: detecting, at the computing device, based on the received data corresponding to the detected jaw movement, one or more of a direction of movement, a duration of movement, a distance of movement, a duration between consecutive movements in the same direction, a time gap between movements in the opposite directions.

In an embodiment, the method may further include the step of: detecting, based on the detected one or more of a direction of movement, a duration of movement, a distance of movement, a duration between consecutive movements in the same direction, a time gap between movements in the opposite directions, at least one of a swallowing action and a food intake action of the user.

In an embodiment, the method may further include the step of: providing a warning when it is detected that the number of chewing actions between consecutive food intake actions, or between a food intake action and a swallowing action is less than a predefined numbers.

In an embodiment, the method may further include the step of: providing an output in respect of historical data in respect of the number of chewing actions between consecutive food intake actions, or between a food intake action and a swallowing action.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 illustrates an exemplary environment of the disclosed system for monitoring chewing habit of a person, in accordance with embodiments of the present disclosure.

FIG. 2 illustrates an exemplary block diagram of the proposed system for monitoring chewing habit of a person, in accordance with embodiments of the present disclosure.

FIG. 3 illustrates an exemplary flow diagram for the disclosed method for monitoring chewing habit of a person, in accordance with embodiments of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

Various terms are used herein. To the extent a term used in a claim is not defined, it should be given the broadest definition persons in the pertinent art have given that term as reflected in printed publications and issued patents at the time of filing.

Embodiments explained herein relate to systems and methods for monitoring chewing habit of a person. In an embodiment, the chewing habit is monitored based on jaw movement. In another embodiment, the jaw movement is detected by a motion sensing device, which is configured as a patch, placed on the face of the person, such as at a lower chin portion of the face of a user to detect movement of lower jaw of the user. In yet another embodiment, the patch includes an accelerometer and/or a gyro-sensor to detect movement of the jaw. The data related to the jaw movement captured by the accelerometer and/or a gyro-sensor is processed by a processor to monitor the chewing habit.

In an embodiment, the processor analyses the data to extract information related to pattern of the jaw movement, and the detected pattern is in turn used to ascertain if the movement is one of a chewing action or a swallowing action, or a food intake action.

For example, the pattern of movement can include a distances moved during a downward movement of lower jaw and an upward movement of the lower jaw, time gap between consecutive downward movement and the upward movement, or between the upward movement and the downward movement. Further, a movement detected after a prolonged absence of movement can be construed to be downward movement of the lower jaw, and subsequent rhythmic alternate movements may be construed as upward and downward movements. A series of rhythmic upward and downward movements can be construed as chewing actions. A prolonged absence of movement after a downward movement followed by a series of upward and downward movements can be construed as a food intake action, and likewise a prolonged absence of movement after an upward movement after a series of upward and downward movements can be construed as a swallowing action. Furthermore, a larger than normal distance covered during downward movement followed by delayed upward movement can be construed as a food/morsel intake action. Based on the ascertained actions, the processor can determine number of chewing actions made by the user between consecutive food intake action and swallowing action.

In an embodiment, the processor can be configured in a portable personal computing device of the user. The patch can include a wireless transmitter to transmit data related to the jaw movement, which can be received by the processor through a wireless transmitter of the computing device or one configured with the processor.

In an embodiment, the processor can be configured to provide, based on the determined chewing actions, feedback to the in respect of chewing habit, such as by providing a warning when it is detected that the number of chewing actions between consecutive food intake actions, or between a food intake action and a swallowing action is less than a predefined numbers, and by providing historical data in respect of the number of chewing actions between consecutive food intake actions, or between a food intake action and a swallowing action, thereby enabling the user to improve his chewing habit and also monitor improvement thereof over a period of time.

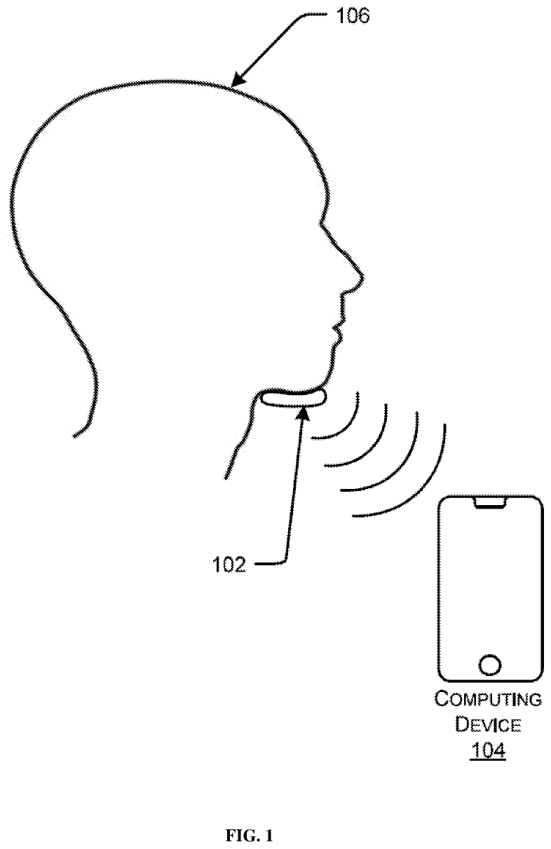

Referring to FIG. 1, where an exemplary schematics of the disclosed system for monitoring chewing habit is shown, the system for monitoring chewing habit 100 (also referred to simply as system 100, hereinafter) can include a motion sensing device 102 (also referred to as a patch 102, hereinafter) configured to be affixed, such as by an adhesive layer provided on the patch 102, to exterior of face of a user 106, such as at a lower chin portion of the user, as shown therein; and a processor configured in a computing device 104. As is evident, the patch 102 shall undergo up and down movements along with the chin as lower jaw of the user 106 is moved for chewing. The patch 102 can be configured to capture data related to the movement and wirelessly transmit the captured data to the computing device 104. The device 104 can include a wireless receiver to receive the transmitted data related to the movement of the jaw. The computing device 104 can process and analyse the received data to determine if the detected jaw movement pertains to a chewing action. The device can thereafter, determine, based on the analysis of the data, number of consecutive chewing actions for each morsel by the user; and provide a feedback to the user in respect of his chewing habit.

In an embodiment, the functionalities of the system 100 can be configured as an app in the computing device 104, and the app can be configured to provide a warning, such as by a buzzer, when it is detected that the number of chewing actions for any of the morsels is less than a predefined numbers. The app can also be configured to provide, such as by displaying on a display screen of the computing device 104, historical data in respect of the average number of chewing actions for morsels for each meal, time taken to complete the meal etc., on different days for a period of time, thereby enabling the user to improve his chewing habit and also monitor improvement thereof over a period of time

As can be seen, keeping the motion sensing device 102 separate from the processor and display, which are configured in the computing device, and coupling the two by means of a low power wireless communication means, such as one of Bluetooth, WiFi, or NFC, can keep the weight of the patch/motion sensing device 102 low.

It is to be appreciated that though the exemplary illustration of FIG. 1 shows the motion sensing device 102 affixed to a lower chin portion of the user, it may be affixed at any other place also on the face, which results in movement of the motion sensing device 102 during chewing action with similar results, and all such variations are well within the scope of the present disclosure without any limitations whatsoever.

It would be appreciated that the patch/motion sensing device 102 can be of any dimension, size, and form/shape, and while the embodiments of the present disclosure indicate it to be positioned on the cheek of a user/subject, it can be positioned on the chin or any other position/place on the face that can allow detection of the jaw/mouth/teeth movement.

In an aspect, the present disclosure can pertain to a motion sensor based device (MSD) configured to measure jaw movements of a user so as to enable evaluation of the chewing practice/pattern of the user at each iteration as well as over a period of time. The MSD can further be configured to enable, through a remote computing device, run a machine learning algorithm to learn the chewing pattern of the user over a period of time and compare it with one or more instances to determine change in pattern/behaviour and give one or more recommendations to the user such as to reduce the speed of chewing, chew more number of times, take smaller bites, reduce gulping of food, among other like recommendations, all of which are well within the scope of the present invention.

In an aspect, the MSD can include or be coupled with a motion sensor, such as an inertial sensor like an accelerometer or a gyroscope. Accelerometers can be used for measuring linear acceleration, and gyroscopes can be used for measuring angular velocity of a object/target article/part. MSD of the present invention can further be coupled to or associated with a Global positioning system (GPS) and location based service (LBS) application to enable determination of an accurate location of the MSD, and motion sensors can often be needed when a GPS signal is attenuated or unavailable, or to enhance the accuracy of GPS location finding.

In an aspect, the proposed MSD can include a deformable substrate configured to conform to a body portion of an individual subject/user; a sensor assembly coupled to the deformable substrate, the sensor assembly including a motion sensor and a physiological sensor, the sensor assembly configured to generate one or more sense signals based on detection of a movement of the body portion by the motion sensor and at least one physiological parameter of the body portion by the physiological sensor during a motion regimen executed by the individual subject; a processor operably coupled to the sensor assembly and configured to receive the one or more sense signals, the processor including circuitry configured to identify a chewing state of the individual subject based on at least one of the movement of the body portion such as mouth or jaw of the user.

In an aspect, the substrate layer can facilitate transfer of epidermal electronics device to an attachment surface, wherein, for instance, the substrate layer may provide a backing which is used to transfer electronics layer to the attachment surface. Substrate layer may then peel away from electronics layer leaving electronics layer attached to attachment surface via a barrier layer, for instance. Substrate layer may also provide protection to electronics layer during the handling of epidermal electronics device. Substrate layer also provides support for electronics layer. Barrier layer can be an elastomer or polymer suited for use in contact with organic tissue. In some embodiments, the barrier layer can be a bio compatible or otherwise inert material. In some embodiments, barrier layer may have a low elastic modulus, e.g., one which is significantly lower (e.g., less than half) of the elastic modulus of attachment surface. For example, the barrier layer may comprise a low modulus polymeric material such as PDMS or BASF. For example, substrate layer 105 may be a rubber or silicone material. In some embodiments, substrate layer may be water soluble. Substrate layer may be dissolved following transfer of the epidermal electronics device onto the attachment surface. In some embodiments, substrate layer need not be biocompatible as it is removed completely or partially following the transfer of epidermal electronics device onto the attachment surface. Substrate layer provides protection to electronics layer from external sources of damage. External sources of damage may include moisture, physical damage (e.g., from a user touching epidermal electronics device), electrical interference, magnetic interference, etc.

In an aspect, the electronics layer can be located between substrate layer and barrier layer. Barrier layer and/or substrate layer can provide support for the elements of electronics layer. In one embodiment, electronics layer can include an array of cells that can contain individual sensors or components. Cells can also be in communication with other components in electronics layer. In some embodiments, cells may be in communication with each other or a subset of other cells within epidermal electronics device. Cells may also be in communication with other elements. For example, cells may be in communication with a power supply, control circuit, and/or communications device. Cells may also contain connections to allow power delivery to the component in the cell, input/output to and from the component in the cell, and/or multiplexing circuitry. In some embodiments, cells may contain sensors such as accelerometers, inclinometers, magnetometers, or gyroscopes. These sensors may be of the micro electro-mechanical systems (MEMS) type, given the small scale of epidermal electronics device and associated components; MEMS accelerometers, gyroscopes, and inclinometers are commercially available from multiple vendors. The sensors may also be part of or supported by integrated circuits or systems on a chip (SOCs). Cells may also contain interaction devices such as drug delivery systems, electrodes, motion capture markers, etc. Interaction devices may also be MEMS, part of or supported by integrated circuits, or SOCs. According to various alternative embodiments, cells may include circuitry facilitating multiplexing of sensor output, transformers, amplifiers, circuitry for processing data and control signals, one or more transistors, etc.

In an exemplary aspect, MSD of the present invention can have an internal capability of give light feedback to the user during the chewing itself, say through a short vibration signal indicating that the user is chewing faster than he should be and that he should talk less or not talk during the chewing, or that he's gulping the foods in less than 15 chews per bite instance.

In an aspect, the proposed MSD can include two sensors that can be arranged to measure the motion (angular and/or translational) of jaw movement. One sensor can be a single-axis accelerometer that can be positioned with its axis of measurement parallel to and along the Z axis of a three dimensional space. Single-axis accelerometer can have a first angle defining a zero degree angle with axis Z. Second sensor can be a second single-axis accelerometer with its axis of measurement not in alignment with the axis Z. Second accelerometer can have an axis of measurement defined by second angle from the Z axis. This angle may be greater than zero degrees. The measurement axis of second single-axis accelerometer is further defined by angle which defines the measurement axis relative to the X-Y plane. As is shown in the illustrated embodiment, single-axis accelerometers can be configured to be slightly opposed (e.g., single-axis accelerometer can be aligned with the Z axis and second single-axis accelerometer is positioned with second angle of thirty degrees and angle of fifteen degrees). In some embodiments, multiple single-axis accelerometers can be configured to measure acceleration along the X, Y, and Z axes. In further embodiments, additional single-axis gyroscopes are configured to measure rotation about the X, Y, and Z axes in addition to acceleration along the X, Y, and Z axes.

In general terms and with reference to the instant disclosure, sensors (e.g., accelerometers, inclinometers, gyroscopes, etc.) are positioned and oriented within electronics layer of epidermal electronics device such that angular motion and orientation of the device may be measured. Many configurations are possible and the embodiments described herein are not intended to be limiting. By using opposed or slightly opposed single-axis sensors of the types discussed, epidermal electronics device may be configured to measure the orientation and/or angular motion of the device and therefore the attachment surface to which the epidermal electronics device is attached.

In an exemplary aspect, MSD of the present disclosure can include or be coupled to a sensor device for analyzing jaw movement of a user, at least one flexible substrate operable to attach to a portion of the user, and a power source embedded on or in the at least one flexible substrate and operable to power the sensor device. In an aspect, the device can further include or be coupled to at least one memory device storing microprocessor executable instructions embedded on or in the at least one flexible substrate, and a microprocessor embedded in or within the at least one flexible substrate, communicatively coupled to the at least one memory device, and executing the microprocessor executable instructions. In an aspect, at least one sensor device can be embedded on or in the at least one flexible substrate and operable to obtain at least one measurement of mouth/jaw movement of the user, wherein through a wireless communication element embedded on or in the at least one flexible substrate and operable to transmit data indicative of the at least one measurement obtained by the at least one sensor. In an aspect, the sensor device can include a plurality of flexible interconnects for electrically connecting wireless communication components, wherein the at least one sensor device can include any or a combination of an accelerometer and/or a gyroscope.

In yet another aspect, the MSD can include a sensor device including a pressure sensor that measures pressure at one or more portions of the mouth/jaw on a subject/user; a memory that stores the user variables including the location of the pressure sensor on the subject/user; and a data processor that receives pressure data from the pressure sensor, the data processor being configured to compute an accumulated pressure value during one or more time periods and, based on the computed accumulated pressure value and the location of the sensor device on the patient, compute one or more data outputs indicative of chewing habits and practices of the user in order to give recommendations to the user on how to make food chewing more effective.

In an aspect, the present disclosure relates to a device that is removably affixed to an exterior part of face of a user, said device being configured to, through motion sensing, detect jaw movement of said user while said user chews a food item, and transmit one or more data packets corresponding to the detected jaw movement to a remote device. The remote device can be a computing device configured to process the received one or more data packets and evaluate, for said user, any or a combination of chewing pattern, chewing frequency, number of chews, timespan of chewing associated with one or more bites of said food items. The remote device can further be configured to implement a machine learning algorithm to evaluate chewing trends of said user and/or of a plurality of other users over a duration of time to indicate one or more chewing recommendations to the user.

Machine learning algorithm(s) can be characterized by a learning style including any one or more of: supervised learning (e.g., using logistic regression, using back propagation neural networks), unsupervised learning (e.g., using an Apriori algorithm, using K-means clustering), semi-supervised learning, reinforcement learning (e.g., using a Q-learning algorithm, using temporal difference learning), and any other suitable learning style. Furthermore, the machine learning algorithm can implement any one or more of: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial lest squares regression, Sammon mapping, multidimensional scaling, projection pursuit, etc.), an ensemble method (e.g., boosting, boostrapped aggregation, AdaBoost, stacked generalization, gradient boosting machine method, random forest method, etc.), and any suitable form of machine learning algorithm.

In an aspect, the proposed invention uses of a modified physiological model of the user, which is trained using the results of measuring the user's jaw movement and the output is a calculated individual response of the user to a particular food taken. The data on the individual response of the user's body is used as auxiliary data for training the machine learning algorithm used to estimate the meals time and classifying food intakes over one or several days. In particular, convolutional neural networks, recurrent neural networks as well as methods of mathematical statistics or other known methods of machine learning can be used as a machine learning algorithm. The measured user's daily activity parameters, including the results of measuring the mouth/jaw movement level of the user are inputted in such a machine learning algorithm for training it as well as auxiliary data for training, namely, data on the individual response of the user's body.

According to another possible embodiment, the system for providing recommendations for maintaining a healthy lifestyle basing on user's daily activity parameters is further configured to receive manually inputted data from the user regarding the user's daily activity parameters, for example, manually inputted names of the food taken or downloading photos of the food taken. In particular, the user can manually input the required parameters when using the device for the first time to specify the initial calibration of the computational physiological model for this particular user. The processing unit, in its turn, is configured to implement said algorithm, including the analysis of data inputted by the user (for example, determining the calorie content of food inputted by the user, or recognizing food in the user's photo and the subsequent determining its calorie content).

The estimation results of the meals time and classification of food intakes, obtained using the machine learning algorithm according to the present invention, are compared with the calibration result of the computational physiological model, and the comparison result is used to refine the estimation of nutrition parameters, i.e. the physiological model calculates the expected response to the amount of food calculated by the algorithm, this expected response is compared with the real response of the user's body and, if they diverge crudely, the algorithm recalculates the amount of food (training of the algorithm with real responses being accumulated over a certain period of time-from several hours to a few days). Thus, the accuracy of estimation of the meals time and classification of food intakes is improved, by taking into account a physiological model calibrated for a particular user. If necessary, the user can correct manually the estimated meals time and sleep time.

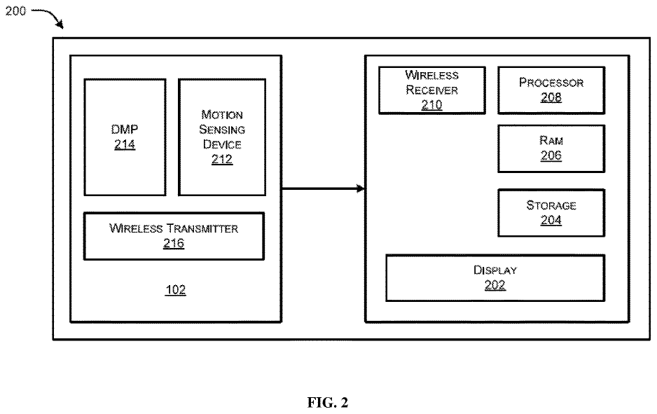

Referring to FIG. 2, the motion sensing device can include one or more of sensors useful in detection of motion of lower part of chin while chewing. In an aspect, the sensors can be any or a combination of a 3-axis accelerometer and a 3-axis gyroscope to give a combined 6-axis motion data on both linear velocity and angular rotation of moving portion of face. The sensors 212 can be fabricated on a single board.

In another aspect, the motion sensing device 102 can include a digital motion processor 214, hereafter referred by the abbreviation DMP, which can read analogue data from the sensors 212 and converts it into suitable digital signal for transmission.

In another aspect, the motion sensing device 102 can include a wireless transmission means 216 for relaying data from the sensors 212 to the computing device 104.

In an embodiment, the computing device 104 can include a processor 208, at least one random access memory device 206, also referred by the abbreviation RAM, at least one storage means 204, at least one display 202 and a wireless receiver 210. In addition, the computing device 104 can include other additional features that are not immediately useful for core operation of the system described in this invention.

In an embodiment, the computing device 104 can be any of a smart phone, a smart tablet, a smart watch, a fitness tracker, a smart wearable, and a laptop, which in the contemporary world, people have ready access to. These are also equipped with means for wireless communication including at least one of, and sometimes all three of, Bluetooth, WiFi and NFC. These devices usually have a display as well as internal processing hardware, including memory and data storage means, as well software to control all these features.

In an embodiment, where the user may like to use more than one computing device, the motion sensing device 102 may wirelessly communicate with more than one computing device. Thus, wireless communication of the motion sensing device 102 can be one-to-one or one-to-many computing devices.

In an embodiment, the processor 202 can be coupled with the memory 206, which can store one or more instructions executable by the processor 202. The processor 202, based on the execution, can receive from motion sensing device 102, the data packet corresponding to the detected jaw movement. The processor 202 can further determine, from the received data packet corresponding to the detected jaw movement, if the detected jaw movement pertains to a chewing action; and determine, based on the determined chewing action, a number of consecutive chewing actions by the user. Based on the determined number of consecutive chewing actions by the user, the processor 202 can provide a feedback to the user in respect of his chewing habit. The feedback can be in form of a warning when it is detected that the number of chewing actions between consecutive food intake actions, or between a food intake action and a swallowing action is less than a predefined numbers, and by providing historical data in respect of the number of chewing actions between consecutive food intake actions, or between a food intake action and a swallowing action, thereby enabling the user to improve his chewing habit and also monitor improvement thereof over a period of time.

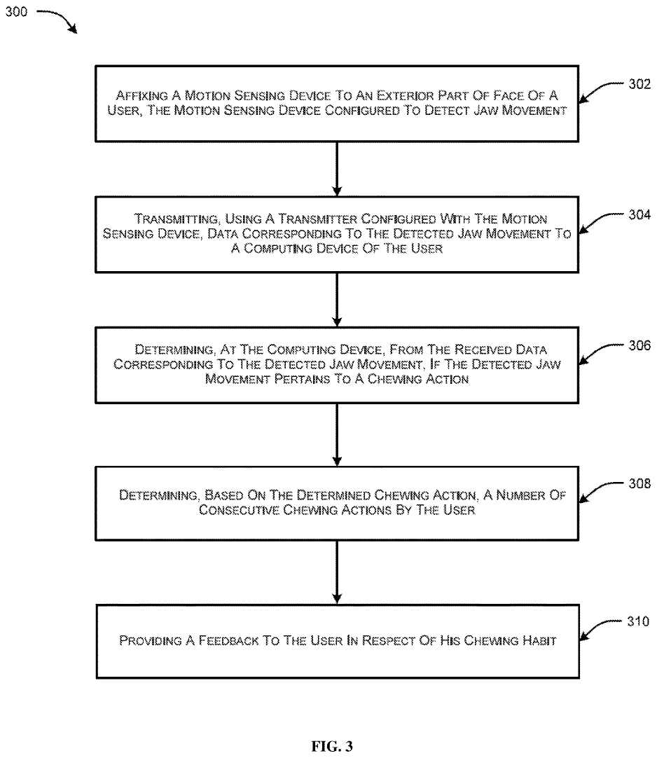

Referring to FIG. 3, the method 300 for monitoring chewing habit of a person (Also referred hereinafter as method 300) can include step of 302 which involves affixing a motion sensing device, such as motion sensor device 102 shown in FIGS. 1 and 2, to an exterior part of face of a user, such as shown affixed to the user 106 in FIG. 1, wherein the motion sensing device 102 can be configured to detect jaw movement. Step 304 of the method 300 can be to transmit, using a transmitter, such as transmitter 216 shown in FIG. 2, configured with the motion sensing device 212, data corresponding to the detected jaw movement to a computing device, such as computing device 104 shown in FIGS. 1 and 2, of the user. At step 306 of the method 300, the computing device 104 can determine from the received data corresponding to the detected jaw movement, if the detected jaw movement pertains to a chewing action. Step 308 of the method 300 can be to determine, based on the determined chewing action, a number of consecutive chewing actions by the user; and 310 can be to provide a feedback to the user in respect of his chewing habit.

In an embodiment, the method 300 can further include the step of: detecting, at the computing device, based on the received data corresponding to the detected jaw movement, one or more of a direction of movement, a duration of movement, a distance of movement, a duration between consecutive movements in the same direction, a time gap between movements in the opposite directions.

In an embodiment, the method 300 can further include the step of: detecting, based on the detected one or more of a direction of movement, a duration of movement, a distance of movement, a duration between consecutive movements in the same direction, a time gap between movements in the opposite directions, at least one of a swallowing action and a food intake action of the user.

In an embodiment, the method 300 can further include the step of: providing a warning, when it is detected that the number of chewing actions between consecutive food intake actions, or between a food intake action and a swallowing action is less than a predefined numbers.

In an embodiment, the method 300 can further include the step of: providing an output in respect of historical data in respect of the number of chewing actions between consecutive food intake actions, or between a food intake action and a swallowing action.

Thus the present disclosure provides a simple and easy to implement system and method for monitoring chewing habit of a person. The disclosed system and method include providing a feedback, as a warning, which can alert a user that he has not chewed the morsel adequately, or as historical data, which can enable the user to assess his progress towards a better chewing habit.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### ADVANTAGES OF THE INVENTION

The present invention provides provide a simple and easy to implement system and method for monitoring chewing habit of a person.

The present invention provides a system and method that helps a user to improve his chewing habit.

The present invention provides a device that helps in detecting chewing action of a user.

The present invention provides a system that helps in detecting number of times a morsel is chewed.

The present invention provides a system that provides an instant feedback to user on any deviation from a desired chewing habit.

The present invention provides a system that provides an feedback on change in his habit over a period of time.

The present invention provides a system that can be implemented with a portable device of the user, such as a smartphone.

We claim:

1. A device (102) configured to be removably affixed to jaw portion of face of a user (106), said device (102) being configured to, through motion sensing, detect jaw movement of said user (106) while said user (106) chews a food item, and transmit one or more data packets corresponding to the detected jaw movement to a remote device (104), wherein said remote device (104) is a computing device that processes the received one or more data packets and evaluate, for said user (106), any or a combination of chewing pattern, chewing frequency, number of chews, timespan of chewing associated with one or more bites of said food item, and wherein pattern of jaw movement is selected from any or a combination of direction of movement, a duration of movement, a distance of movement, a duration between consecutive movements in same direction, and time gap between movements in opposite directions.

2. The device (102) as claimed in claim 1, wherein said remote device (104) implements a machine learning algorithm to evaluate chewing trends of said user (106) over a duration of time to indicate one or more chewing recommendations to the user (106), wherein the chewing trends comprise any or a combination of the evaluated chewing pattern, the chewing frequency, the number of chews, the timespan of chewing associated with one or more bites.

3. A system (100) for monitoring chewing habit, comprising:
a motion sensing device (102) configured to be removably affixed to an exterior part of face of a user (106), the motion sensing device configured to detect jaw movement and transmit a data packet corresponding to the detected jaw movement to a computing device, said computing device comprising a processor coupled with a memory, wherein the memory stores one or more instructions executable by the processor to:
receive, from motion sensing device (102), the data packet corresponding to the detected jaw movement;
determine, from the received data packet corresponding to the detected jaw movement, if the detected jaw movement pertains to a chewing action;
determine, based on the determined chewing action, a number of consecutive chewing actions by the user (106); and
provide a feedback to the user (106) in respect of his/her chewing habit based on the determination of consecutive chewing actions by the user (106), wherein said chewing habit is based any or a combination of chewing pattern, chewing frequency, number of chews, timespan of chewing associated with one or more bites of said food item.

4. The system (100) as claimed in claim 3, wherein the motion sensing device (102) is configured as a patch adapted to be affixed to a lower chin portion of the face of the user (106).

5. The system (100) as claimed in claim 3, wherein the motion sensing device (102) is configured for being removably affixed to the face of the user (106).

6. The system (100) as claimed in claim 3, wherein the motion sensing device (102) comprises any or a combination of an accelerometer and a gyro-sensor to detect the jaw movements. and a wireless transmitter to transmit the data packet corresponding to the detected jaw movement.

7. The system (100) as claimed in claim 3, wherein the processor is configured to detect at least one of a swallowing action and a food intake action of the user (106).

8. The system (100) as claimed in claim 7, wherein the processor is configured to detect the chewing action, the swallowing action and the food intake action based on pattern of the jaw movement.

9. The system (100) as claimed in claim 7, wherein the pattern of jaw movement includes one or more of a direction of movement, a duration of movement, a distance of movement, a duration between consecutive movements in the same direction, a time gap between movements in the opposite directions.

10. The system (100) as claimed in claim 7, wherein the processor is configured to provide a warning when it is detected that the number of chewing actions between consecutive food intake actions, or between a food intake action and a swallowing action is less than a predefined number.

11. The system as claimed in claim 7, wherein the processor is configured to provide an output in respect of historical data in respect of the number of chewing actions between consecutive food intake actions, or between a food intake action and a swallowing action.

12. A method (300) for monitoring chewing habit of a user (106), the method (300) comprising the steps of;
affixing (302) a motion sensing device (102) to an exterior part of face of a user (106), the motion sensing device (102) being configured to detect jaw movement;
transmitting (304), using a transmitter configured with the motion sensing device (102), data corresponding to the detected jaw movement to a computing device of the user (106);
determining (306), at the computing device, from the received data corresponding to the detected jaw movement, if the detected jaw movement pertains to a chewing action;
determining (308), based on the determined chewing action, a number of consecutive chewing actions by the user (106); and
providing (310) feedback to the user (106) in respect of his/her chewing habit based on the determination of consecutive chewing actions by the user (106), wherein said chewing habit is based any or a combination of chewing pattern, chewing frequency, number of chews, timespan of chewing associated with one or more bites of said food item.

13. The method (300) as claimed in claim 12, comprising the step of: detecting, at the computing device, based on the received data corresponding to the detected jaw movement, one or more of a direction of movement, a duration of movement, a distance of movement, a duration between consecutive movements in the same direction, a time gap between movements in the opposite directions.

14. The method (300) as claimed in claim 13, comprising the step of: detecting, based on the detected one or more of a direction of movement, a duration of movement, a distance of movement, a duration between consecutive movements in the same direction, a time gap between movements in the opposite directions, at least one of a swallowing action and a food intake action of the user (106).

15. The method (300) as claimed in claim 14, comprising the step of: providing a warning when it is detected that the number of chewing actions between consecutive food intake actions, or between a food intake action and a swallowing action is less than a predefined numbers.

16. The method (300) as claimed in claim 15, comprising the step of: providing an output in respect of historical data in respect of the number of chewing actions between consecutive food intake actions, or between a food intake action and a swallowing action.
